(19) United States
(12) Patent Application Publication
(10) Pub. No.: US 2026/0232937 A1
(43) Pub. Date: Aug. 13, 2026

(54) LOW-PROFILE UNIVERSAL RESPIRATORY MASK

(71) Applicant: CM DYNAMIC LLC, Afton, WY (US)

(72) Inventor: Chad Aaron BAGLEY, Afton, WY (US)

(21) Appl. No.: 19/535,975

(22) Filed: Feb. 10, 2026

Related U.S. Application Data
(60) Provisional application No. 63/756,660, filed on Feb. 10, 2025.

(51) Int. Cl. A61M 16/06

(52) U.S. Cl. CPC A61M 16/0616; A61M 2202/0007; A61M 2202/02; A61M 2205/0216; A61M 2210/0618; A61M 2210/0625

(57) ABSTRACT

A respiratory mask is provided, comprising a mask body defining an exterior surface, a perimeter, and an interior cavity configured to cover a patient's nose and mouth. An inflatable ring is attached to the perimeter of the mask body, with the ring comprising a plurality of air chambers sealed from one another and connected to the perimeter by an adhesive material. The mask further includes at least two inlets on opposing lateral sides, configured to deliver or exhaust gases. The inlets may have threaded connections to securely engage with complementary fittings, such as caps or tubes that connect to a breathing circuit. The mask is designed for use with oxygen and anesthetic gas delivery systems and may include a gasket portion made of a thermoplastic elastomer material to provide a seal with the patient's skin. The inflatable ring is designed with overlapping air chambers and independent inflation ports for adjusting the fit of the mask to conform to individual facial proportions. The mask is particularly suitable for use in oxygen and anesthesia delivery applications.

### CROSS-REFERENCE TO RELATED APPLICATIONS

This application claims priority to U.S. Provisional Patent Application No. 63/756,660, filed on Feb. 10, 2025, the entirety of which is incorporated herein by reference.

### BACKGROUND OF THE INVENTION

### 1. The Field of the Invention

The present disclosure relates generally to respiratory masks and, more particularly, to low-profile respiratory masks configured for delivery of oxygen and anesthetic gases in clinical settings, in various patient positions.

### 2. Background and Relevant Art

Anesthesia and oxygen masks are critical tools for maintaining and supporting respiratory function during various medical procedures. These masks typically deliver oxygen or anesthetic gases via a delivery circuit that is connected to the mask via tubing. Many existing masks include a face-contacting body, a gas inlet positioned at an anterior apex of the mask, and downstream components such as filters, elbows, and breathing circuits that extend outward from the patient's face.

Such anteriorly projecting configurations increase the overall profile of the mask apparatus and can interfere with patient positioning, clinician access, and sealing effectiveness. In particular, when patients are positioned prone or semi-prone, anterior mask components may contact a supporting surface, resulting in unintended forces applied to the patient's face, compromised sealing, and increased risk of pressure-related injury.

Additionally, many conventional respiratory masks rely on uniform peripheral cushions or rigid flanges that are unable to accommodate asymmetrical facial anatomy. Variations such as prominent nasal bridges, recessed chins, or uneven facial contours can result in localized gaps or pressure concentrations, requiring increased manual force to maintain a seal. This can lead to discomfort, inconsistent gas delivery, and clinician fatigue.

Accordingly, there exists a need for a respiratory mask that reduces anterior profile, accommodates varied facial anatomy without excessive force, and facilitates reliable sealing during manual use, including in prone or non-supine positions.

### BRIEF SUMMARY

In accordance with one or more embodiments, a low-profile respiratory mask is provided that includes a mask body defining an interior cavity configured to cover a patient's nose and mouth, and an inflatable perimeter structure secured to the mask body. The inflatable perimeter structure includes a plurality of isolated air chambers that may be independently inflated to conform to individual facial contours.

In some embodiments, the mask body includes at least two gas inlets positioned on opposing lateral portions of the mask body, thereby reducing anterior projection of connected gas delivery components. One inlet may be selectively capped while another is coupled to a gas delivery or exhaust system.

In some embodiments, the air chambers are separated by partitions oriented such that adjacent chambers overlap along a non-orthogonal boundary, reducing pressure discontinuities at chamber transitions. A compliant gasket material may be disposed over at least a portion of the inflatable perimeter to enhance sealing with minimal applied force.

Additional features and advantages of exemplary implementations of the present invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. For better understanding, the like elements have been designated by like reference numbers throughout the various accompanying figures. Understanding that these drawings depict only typical embodiments of the invention and are not therefore to be considered to be limiting of its scope, the invention will be described and explained with additional specificity and detail through the use of the accompanying drawings in which:

FIG. 1 illustrates a side perspective view of a mask connected to a gas flow system that is exemplary of the masks currently in widespread use.

FIG. 2A illustrates an embodiment of the mask disclosed herein, showing its connection to a gas flow system via inlets located on a lateral surface of the mask exterior. FIG. 2B discloses an alternative embodiment of the current disclosure, illustrating an exploded view of the air chamber and the seal.

FIG. 3 illustrates a view of the exterior surface of the mask of the current disclosure, including two threaded inlets and inflation ports, as well as a cap that can optionally seal one of the inlets.

FIG. 4A illustrates a lateral perspective view of the mask body of the current disclosure. FIG. 4B illustrates a lateral perspective view of an alternative embodiment of the current disclosure, including a capped inlet disposed on the apex of the mask body in addition to the inlet on the lateral surface.

FIG. 5 illustrates a side perspective view of a patient using a mask of the prior art where there are no inflatable air chambers.

FIG. 6 illustrates a side perspective view of a patient using a mask of the current disclosure, with inflatable air chambers facilitating a custom fit.

FIG. 7 illustrates a side perspective view of a current mask in use on a patient in the prone position.

FIG. 8 illustrates a side perspective view of a mask of the current disclosure being used on a patient in the prone position.

### DETAILED DESCRIPTION

### Overview

The following detailed description describes various embodiments of a low-profile respiratory mask configured to deliver oxygen, anesthetic gases, or ventilatory support to a patient while improving maneuverability, sealing performance, and patient comfort. The disclosed embodiments address clinical needs associated with maintaining an effective facial seal under manual application, including during non-supine patient positioning such as prone, lateral, or semi-prone orientations.

The present disclosure relates to a respiratory mask including a mask body defining an interior cavity sized to cover a patient's nose and mouth, and an inflatable perimeter structure secured to the mask body. The inflatable perimeter structure includes a plurality of isolated air chambers that may be independently inflated to conform to individual facial anatomy, thereby facilitating a secure seal with reduced applied force.

In some embodiments, the mask body includes at least two gas inlets positioned on opposing lateral portions of the mask body. This configuration enables lateral routing of connected filters, adapters, and breathing circuits, reducing anterior projection of the mask apparatus and minimizing interference with supporting surfaces when the patient is positioned prone or otherwise non-supine.

The disclosed embodiments may include a compliant gasket or sealing layer disposed over at least a portion of the inflatable perimeter structure. The gasket may be formed from soft, flexible materials, including thermoplastic elastomers or heavily plasticized polymer materials, and is configured to conform to patient facial features under light manual pressure while reducing localized pressure and the risk of skin injury.

The inflatable perimeter structure may include multiple air chambers separated by flexible partitions. In some embodiments, adjacent air chambers overlap along non-orthogonal boundaries, which can reduce abrupt pressure transitions between chambers and improve comfort when different regions of the mask are inflated to different pressures.

The disclosed respiratory mask is configured for use without reliance on head straps or rigid fixation, allowing a healthcare provider to manually maintain the mask against the patient's face while preserving access and control of the airway. This configuration can reduce setup complexity and improve responsiveness during anesthesia delivery, oxygen supplementation, or rescue ventilation.

The figures and accompanying description illustrate exemplary configurations and uses of the disclosed respiratory mask. These examples are provided for purposes of illustration and explanation and are not intended to limit the scope of the claimed subject matter. Variations in materials, inlet configurations, air chamber arrangements, and sealing structures consistent with the disclosed embodiments may be made without departing from the scope of the disclosure.

In some embodiments, the respiratory mask may be used as part of a broader respiratory or patient positioning system, including anesthesia delivery systems, ventilators, filters, adapters, and prone positioning supports. The mask may function as a reusable or disposable component suitable for use across a variety of clinical applications, including surgical, procedural, emergency, and critical care settings.

### Example Respiratory Mask

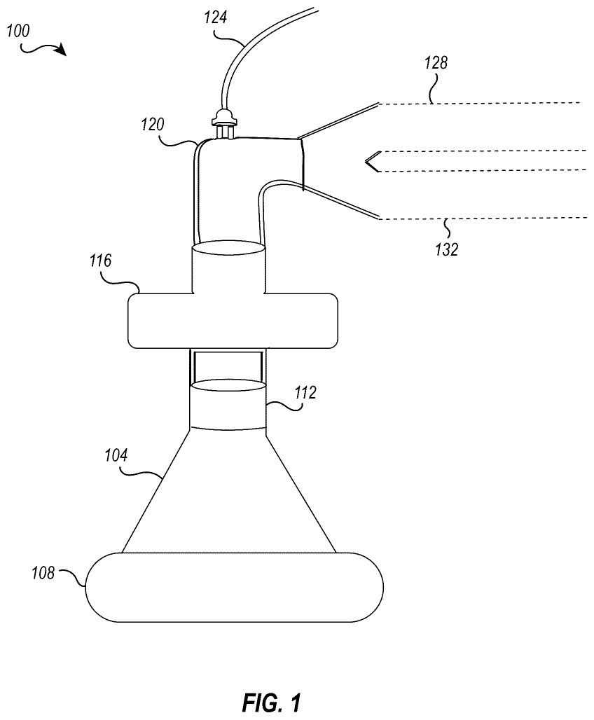

FIG. 1 illustrates a respiratory mask apparatus 100 representative of conventional prior art configurations commonly used for oxygen delivery and anesthesia administration. The illustrated apparatus includes a mask body 104 defining an interior cavity configured to cover a patient's nose and mouth. The mask body 104 includes an anteriorly positioned apex from which an orifice 112 extends outwardly along a longitudinal axis generally perpendicular to the patient's face. In some conventional mask configurations, such as the mask apparatus 100 illustrated in FIG. 1, a peripheral air cushion 108 may be disposed around the perimeter of the mask body 104, the air cushion being configured to contact a patient's face to assist in forming a seal, although such cushions may provide limited adaptability to varied facial anatomy.

The orifice 112 is configured to receive a filter 116, which in turn is coupled to a rigid elbow 120. The elbow 120 connects to oxygen tubing 124 and associated gas delivery circuits, including an inspiratory limb 128 and an expiratory limb 132. Collectively, these components extend directly outward from the anterior apex of the mask body 104, resulting in a high-profile configuration.

In typical implementations, the combined length of the orifice 112, filter 116, elbow 120, and associated tubing 124 produces a total vertical projection that can approach or exceed approximately seven inches when measured from the patient-facing perimeter of the mask body 104 to the distal portion of the connected circuitry. This protruding geometry increases the clearance envelope required in front of the patient's face during use.

Such anterior projection can interfere with patient positioning, particularly when the patient is placed in a prone or semi-prone orientation. In these positions, the projecting components may contact a supporting surface such as an operating table or hospital bed, causing reactive forces to be transmitted back through the mask apparatus 100 toward the patient's face.

The resulting forces may compromise the seal between the mask body 104 and the patient's facial skin, increase localized pressure at the mask-to-face interface, and necessitate additional manual force by a healthcare provider to maintain an effective seal. These effects can contribute to patient discomfort, inconsistent gas delivery, and an increased risk of pressure-related injury during extended procedures.

The configuration illustrated in FIG. 1 further limits the ability of a clinician to reposition or maneuver the mask apparatus 100 while maintaining airway access, particularly when manual jaw thrust or airway stabilization is required. These limitations underscore the need for alternative respiratory mask configurations with reduced anterior profile and improved maneuverability, as described in the embodiments that follow.

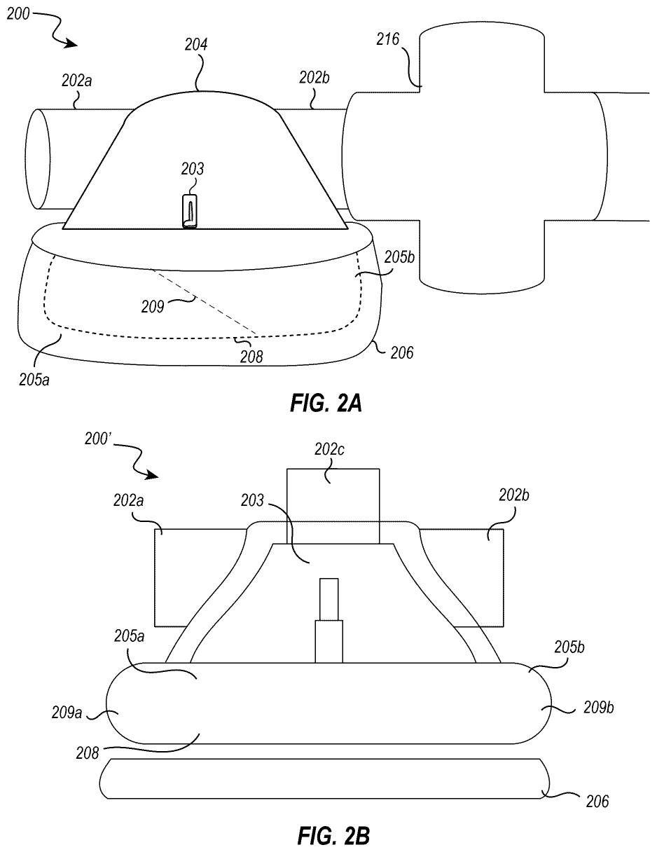

FIG. 2A illustrates an exemplary respiratory mask 200 having a low-profile configuration. The mask 200 includes a mask body 204 defining an exterior surface, an interior cavity configured to receive a patient's nose and mouth, and a perimeter configured to engage a patient's face during use. The mask body 204 includes an apex that may define a maximum anterior projection of the apparatus. In some embodiments, the overall vertical height of the mask 200, measured from a patient-facing perimeter to the apex of the mask body 204, may be less than approximately three inches, although greater or lesser heights are contemplated depending on component selection, intended use, and manufacturing considerations.

The mask body 204 may include at least two gas inlets 202a and 202b positioned on opposing lateral surfaces of the exterior surface. The lateral positioning of the inlets 202a, 202b may permit gas delivery components to be routed laterally rather than anteriorly, which can reduce forward projection of the apparatus and improve maneuverability. In some embodiments, this configuration may facilitate ambidextrous use, allowing a healthcare provider to connect a filter, adapter, or breathing circuit on either side of the mask depending on preference or spatial constraints.

In the illustrated embodiment, a filter 216 and associated breathing circuit components (not shown) are coupled to inlet 202b. However, it should be understood that the filter 216 and breathing circuit components may alternatively be coupled to inlet 202a, or that both inlets may be used simultaneously in certain configurations. One or more unused inlets may be sealed using a cap or other closure to prevent gas leakage during use. The mask body 204 may be coupled to an inflatable ring 208 disposed along at least a portion of the perimeter of the mask body. The inflatable ring 208 may be secured to the mask body 204 using an adhesive material, which can include silicone-based adhesives, pressure-sensitive adhesives, polyurethane-based adhesives, or other suitable bonding agents. In some embodiments, the inflatable ring 208 may be permanently affixed, while in other embodiments it may be replaceable or removable.

The inflatable ring 208 may comprise a plurality of air chambers 205a, 205b that are sealed from one another such that inflation of one chamber does not result in gaseous communication with adjacent chambers. The air chambers 205a-b may be formed from flexible, deformable materials including polyvinyl chloride, silicone, thermoplastic elastomer polymers, rubber, or combinations thereof. One or more partitions 209 may separate adjacent air chambers, and such partitions may be oriented diagonally, obliquely, or otherwise offset relative to a vertical or horizontal axis of the mask body. This orientation may cause adjacent air chambers to partially overlap along their boundaries.

FIG. 2B illustrates an alternative embodiment 200′ of the mask 200. In this embodiment, the mask body 204 further includes an inlet 202c positioned near the apex of the mask body. The apex inlet 202c may permit use of conventional anteriorly routed filters and breathing circuits if desired, while still allowing use of the lateral inlets 202a, 202b. In some embodiments, any one of the inlets 202a, 202b, or 202c may be selectively coupled to a filter 216 or breathing circuit components, and any remaining inlets may be capped or otherwise sealed during use.

In the embodiment illustrated in FIG. 2B, the inflatable ring 208 may include at least two air chambers 205a and 205b separated by partitions 209a and 209b extending across a width of the inflatable ring. The partitions may be configured to define sealed air chambers that are not in gaseous communication with one another. The partitions 209a, 209b may be oriented diagonally or non-orthogonally such that portions of adjacent air chambers overlap, although other configurations are also contemplated. FIG. 2B further illustrates an exploded view of the inflatable ring 208 and a seal 206 disposed over at least a portion of the inflatable ring. The seal 206 may be configured to cover only a patient-facing portion of the inflatable ring, although in other embodiments the seal may extend around a greater portion of the ring. The seal 206 may be formed from a thermoplastic elastomer polymer (TEP) material, including gel-based or elastomeric formulations selected to provide a soft, compliant interface with a patient's skin.

In alternative embodiments, the seal 206 may be additionally or alternatively formed from other deformable materials capable of conforming to facial anatomy under light manual pressure, including plasticized polymer materials, silicone gels, polyurethane gels, elastomeric foams, or combinations thereof. The seal material may be selected based on functional characteristics such as softness, tack, resilience, recovery, durability, and biocompatibility rather than a specific chemical composition. The seal 206 may exhibit a Shore 00 hardness of up to approximately 00-10, and in some embodiments between approximately 00-0 and 00-5, although harder or softer constructions are contemplated. Certain embodiments may utilize materials whose compliance is better characterized using compression, indentation, or force-deflection testing rather than Shore hardness alone. The seal 206 may be configured to conform under light manual pressure, maintain sealing engagement during minor patient movement, and resist leaving residue on the patient's skin.

The overlapping arrangement of the air chambers 205a, 205b may reduce abrupt transitions in pressure across the patient's face when adjacent chambers are inflated to different pressures. Each air chamber may correspond to a separate inflation port 203, permitting independent inflation or deflation of individual chambers. Such independent adjustment may allow the mask to accommodate a wide range of facial geometries while reducing the amount of manual force required to maintain an effective seal.

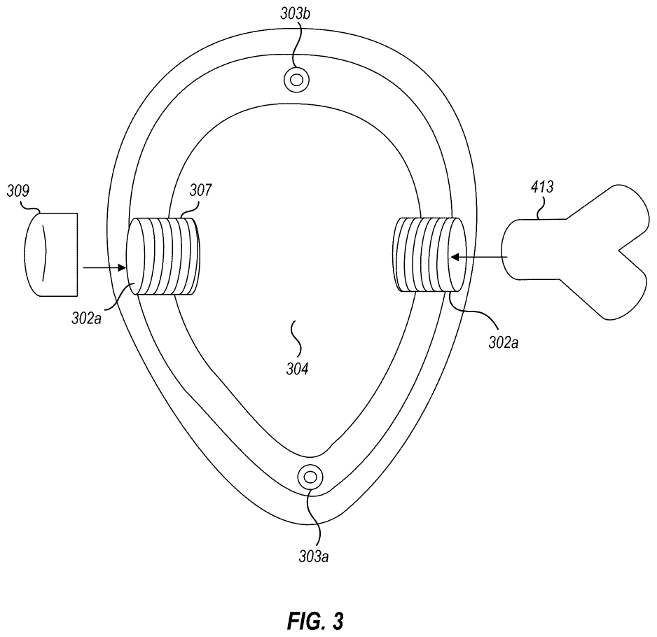

FIG. 3 illustrates an anterior view of a mask body 304 according to embodiments of the present disclosure. The mask body 304 may define an exterior surface and may include one or more gas inlets 302a and 302b disposed on opposing lateral portions of the mask body. In some embodiments, the inlets 302a, 302b may project outward from the exterior surface to facilitate connection to external components, although flush, recessed, or partially recessed configurations are also contemplated.

In some embodiments, one or more of the inlets 302a, 302b may include external threads characterized by helical grooves 307 formed along an outer surface of the inlet. Such threaded configurations may permit releasable engagement with corresponding components, including caps, adapters, or fittings. In other embodiments, the inlets may include internal threads, friction-fit surfaces, snap-fit features, bayonet-style interfaces, or other coupling structures, either alone or in combination with threaded features.

One or more of the inlets 302a, 302b may be selectively sealed during use. In some embodiments, an inlet may be sealed using a removable cap 309. The cap 309 may include an internal threaded surface (not shown) configured to engage an externally threaded inlet, although alternative configurations are contemplated. For example, the cap may engage via friction fit, snap engagement, elastic deformation, or valve-based sealing structures. The cap 309 may be formed from a rigid, semi-rigid, or flexible material, depending on the desired sealing characteristics.

In some embodiments, one or more of the inlets 302a, 302b may be coupled to an adapter 413 configured to interface the mask body 304 with an external gas delivery system, exhaust system, ventilatory circuit, or other respiratory component. The adapter 413 may include an internal threaded surface configured to engage an external thread of the inlet, although other engagement mechanisms may be used, including friction-fit, bayonet-style, latching, or hybrid connections. The adapter 413 may further include one or more outlet portions configured to connect to tubing, filters, elbows, or breathing circuits.

The engagement between an inlet 302a, 302b and a cap 309 or adapter 413 may be configured to inhibit or reduce unintended gas leakage during use. In some embodiments, the engagement may provide a substantially airtight or leak-resistant connection when properly engaged, although minor leakage may be acceptable depending on application. The degree of sealing may vary based on connection type, material selection, tolerances, and operating conditions. The lateral placement of the inlets 302a, 302b may permit routing of connected components away from an anterior region of the mask body 304, which may reduce forward projection of the overall apparatus and improve maneuverability. In some embodiments, one inlet may be used while another inlet is sealed, while in other embodiments multiple inlets may be used simultaneously for delivery, exhaust, monitoring, or other functions. In some embodiments, the mask body 304 illustrated in FIG. 3 may further include one or more inflation ports 303a and 303b associated with an inflatable perimeter structure, the inflation ports being configured to permit introduction or removal of air or another inflation medium to adjust a volume or pressure of one or more air chambers.

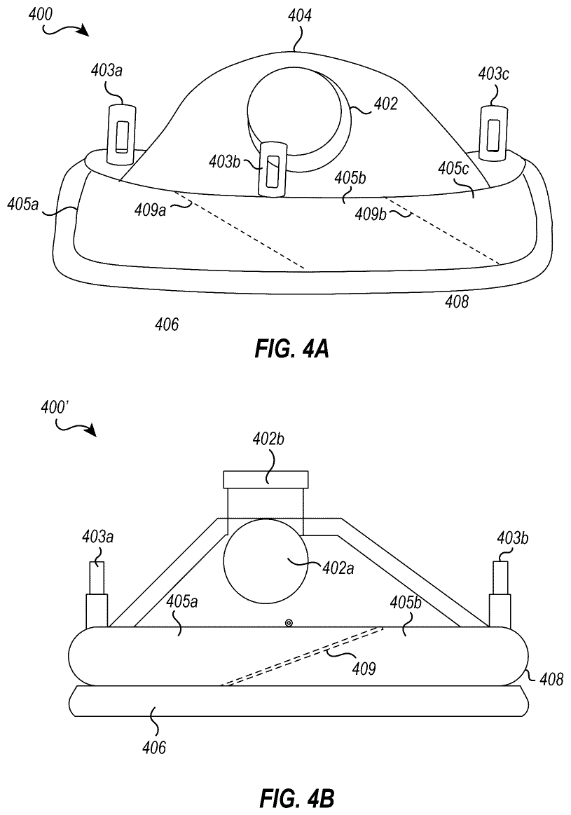

FIG. 4A illustrates a side perspective view of an exemplary respiratory mask 400 according to embodiments of the present disclosure. The mask 400 may include a mask body 404 and an inflatable ring 408 disposed along at least a portion of a perimeter of the mask body. In the illustrated embodiment, the inflatable ring 408 may comprise more than two air chambers, shown as air chambers 405a, 405b, and 405c, although fewer or greater numbers of air chambers are also contemplated depending on desired adjustability and manufacturing considerations.

Each air chamber 405a-405c may include a corresponding inflation port 403a, 403b, 403c configured to permit independent inflation or deflation of the respective air chamber. The inflation ports may be disposed on an exterior surface of the inflatable ring 408 and may be configured for engagement with a syringe, bulb, pump, or other inflation device. Independent inflation of the air chambers may allow selective adjustment of pressure at different regions of the patient-facing perimeter.

The air chambers 405a-405c may be sealed from one another by one or more partitions 409a and 409b. In some embodiments, the partitions may be oriented diagonally, obliquely, or otherwise non-parallel relative to a vertical axis of the mask body 404. Such orientation may cause adjacent air chambers to partially overlap along their boundaries, although other partition orientations and overlap arrangements are also contemplated. The overlapping configuration may reduce abrupt transitions in pressure between adjacent chambers when the chambers are inflated to different pressures.

In the embodiment illustrated in FIG. 4A, the mask body 404 may include one or more gas inlets, such as inlet 402, positioned on a lateral portion of the mask body. The inlet 402 may be configured to form a slip-on connection with a cap, tubing, filter, adapter, or gaseous circuit, although other engagement mechanisms may also be used, including threaded, friction-fit, snap-fit, or hybrid connections.

FIG. 4B illustrates an alternative embodiment 400′ of the respiratory mask 400. In this embodiment, the mask body 404 may include an inlet 402b positioned near an apex of the mask body. The apex inlet 402b may allow the mask to interface with conventional anteriorly routed filters and breathing circuits when desired. In some embodiments, the mask body may further include one or more lateral inlets, such as inlet 402a, such that multiple inlet locations are available for connection. In the embodiment of FIG. 4B, one or more of the inlets 402a, 402b may be configured to attach to a cap, tubing, filter, or gaseous circuit via a slip-on connection. In other embodiments, the inlets may additionally or alternatively include threaded surfaces, friction-fit interfaces, or other coupling features. Any unused inlets may be sealed during use to inhibit gas leakage.

The inflatable ring 408 in the embodiment of FIG. 4B may similarly comprise multiple air chambers, such as air chambers 405a, 405b, and 405c, each associated with a corresponding inflation port 403a, 403b, or 403c. The air chambers may be separated by one or more partitions 409 oriented diagonally or non-orthogonally relative to a vertical axis, such that adjacent chambers may partially overlap. In some embodiments, fewer or greater numbers of chambers may be provided, and the partitions may have alternative orientations or geometries. FIG. 4B further illustrates a seal 406 disposed over at least a portion of the inflatable ring 408. The seal 406 may be configured to cover only a patient-facing portion of the inflatable ring, although in other embodiments the seal may extend around a greater portion or the entirety of the ring. The seal 406 may be formed from a thermoplastic elastomer polymer (TEP) material or other soft, deformable materials as described elsewhere herein, and may be configured to conform to facial anatomy under light manual pressure while maintaining sealing engagement during use.

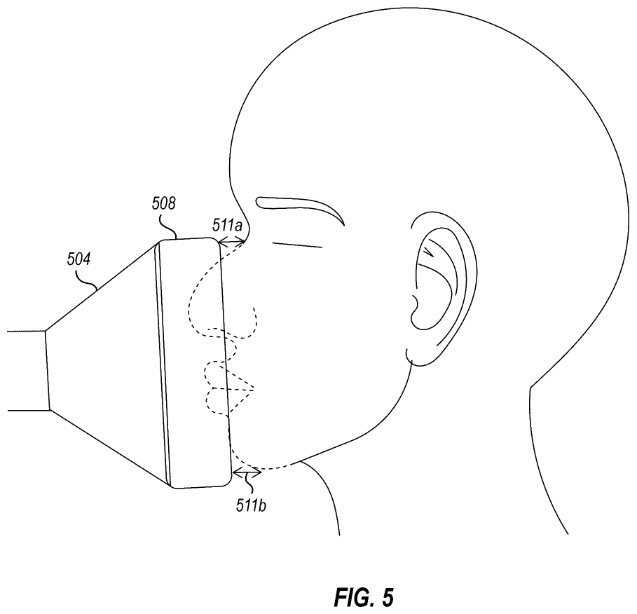

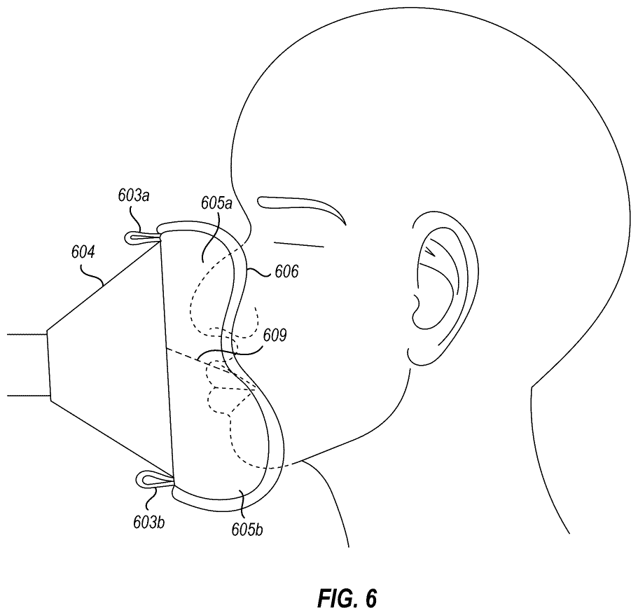

FIGS. 5 and 6 illustrate example interactions between a respiratory mask and a patient's facial anatomy, and demonstrate how independently inflatable air chambers may affect mask fit in certain use conditions. Patients may present with a wide range of facial features, including but not limited to a prominent nasal bridge, a recessed chin, a low nasal bridge, a protruding chin, asymmetrical contours, or combinations thereof. Such anatomical variations may present challenges for achieving a consistent seal using masks that lack localized adjustability.

In FIG. 5, a conventional respiratory mask 504 that does not include independently inflatable air chambers is illustrated in use. In this example, the mask 504 includes a uniform or rigid peripheral sealing structure 508 that may not conform to all facial contours. As a result, one or more gaps 511a and 511b may be present between the mask and the patient's face in certain regions. Such gaps may require increased manual force to close in order to maintain a seal, which in some cases may contribute to patient discomfort or localized pressure. Alternatively, if additional force is not applied, gases delivered through the mask may escape through the gaps, potentially reducing delivery efficiency and increasing exposure to surrounding personnel.

In contrast, FIG. 6 illustrates an example of a respiratory mask 604 according to the present disclosure in use on a patient with similar facial features. In this embodiment, the mask includes an inflatable ring with multiple air chambers, such as chambers 605a and 605b, that may be independently inflated to close the gaps 511a and 511b seen in FIG. 5. The seal(s) 609 between the chambers enable a healthcare provider to inflate nose-adjacent chamber 605a to a lesser degree, as needed, than the chin-adjacent chamber 605b. This ensures that excess pressure is not placed against the nose, and there is not a lack of pressure against the chin. Further, the customizable inflated ring also ensures that seal 606 is in contact with the facial skin of the patient. In some use scenarios, one or more chambers positioned adjacent to the nasal region may be inflated to a different degree than one or more chambers positioned adjacent to the chin or lower facial region. Such differential inflation may allow the mask to conform more closely to the patient's anatomy without requiring uniform pressure across all contact areas.

In some embodiments, the independent adjustability of the air chambers may permit a healthcare provider to reduce pressure applied to more sensitive facial regions while maintaining sufficient pressure in other regions to sustain a seal. The inflatable ring may further cooperate with a patient-contacting seal 606 such that the seal remains in contact with the patient's skin across multiple facial regions, even when individual chambers are inflated to different pressures. This configuration may reduce the likelihood of gaps while limiting localized pressure concentrations.

The inflatable ring may further cooperate with a patient-contacting seal 606 disposed over at least a portion of the inflatable ring. In some embodiments, the seal 606 may remain in continuous contact with the patient's facial skin across multiple regions even when the air chambers 605a and 605b are inflated to different pressures. This configuration may allow the respiratory mask 604 to conform more closely to the patient's facial anatomy without requiring uniform pressure across all contact areas and may reduce the likelihood of gaps between the mask and the patient's face. In some embodiments, the respiratory mask body 604 may further include one or more inflation ports 603a and 603b associated with an inflatable perimeter structure, the inflation ports being configured to permit introduction or removal of air or another inflation medium to adjust a volume or pressure of one or more air chambers.

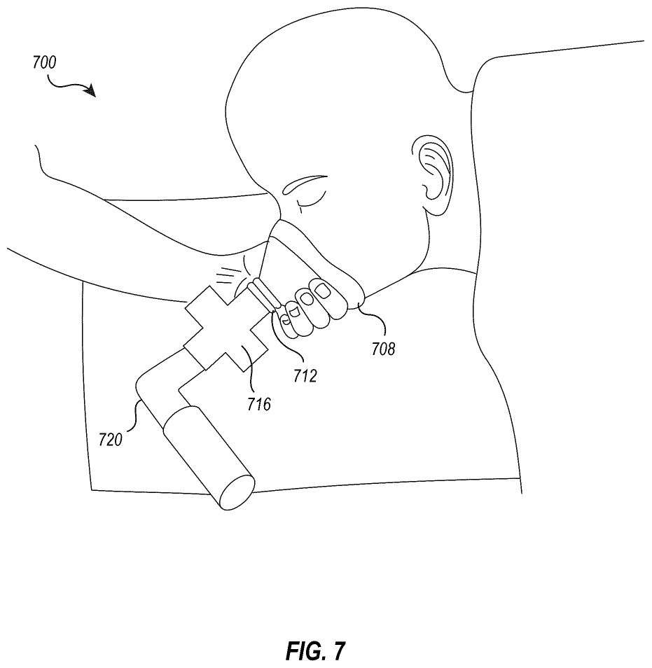

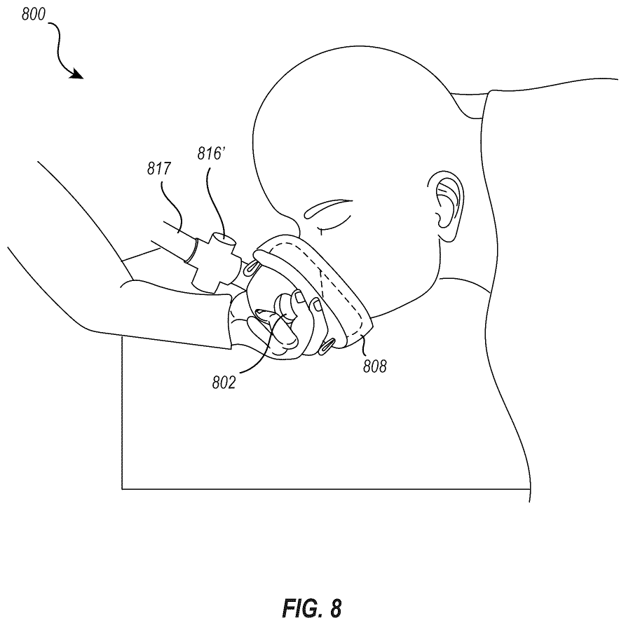

FIGS. 7 and 8 illustrate example differences in use between a conventional high-profile respiratory mask apparatus and a low-profile respiratory mask according to embodiments of the present disclosure, particularly when a patient is positioned prone. Such figures are intended to illustrate potential use scenarios rather than to limit the scope of the disclosure.

FIG. 7 depicts a patient positioned prone while a healthcare provider manually holds a conventional respiratory mask 700 against the patient's face. In some clinical situations, a healthcare provider may also be required to maintain a forward jaw thrust or otherwise support the patient's airway while holding the mask in position. When a patient is positioned prone, the available space between the patient's face and a supporting surface, such as a bed or operating table, may be limited.

In the illustrated example, the conventional mask 700 includes an anteriorly positioned orifice 712 coupled to a filter 716 and a rigid elbow 720 that extend outward from the mask body. This anterior projection may occupy a substantial portion of the available clearance space between the patient's face and the supporting surface. In some scenarios, the projecting components may contact the supporting surface during use.

When such contact occurs, reactive forces from the supporting surface may be transmitted back through the mask apparatus toward the patient's face. These forces may increase localized pressure at the mask-to-face interface and may alter the positioning of the mask relative to the patient's facial anatomy. In addition, conventional masks may include relatively rigid peripheral seals 708 that may not readily accommodate such external forces or facial movement, which in some cases may contribute to patient discomfort or localized pressure effects. Furthermore, in such use scenarios, a healthcare provider may be required to simultaneously manage multiple tasks, including maintaining an airway position, holding the mask in sealing engagement without applying excessive force, accommodating patient movement, and ensuring that connected components such as filters, elbows, and tubing remain properly attached. These combined requirements may increase the complexity of mask use, particularly during prolonged procedures or in constrained environments.

FIG. 8 illustrates an embodiment of a respiratory mask 800 according to the present disclosure being used with a patient in a prone position. In this embodiment, one or more components, such as a filter 816′ and tubing 817, may be coupled to a lateral inlet 802 such that the connected components extend generally to the side of the patient rather than anteriorly. As a result, the connected components may avoid contact with the supporting surface in certain use conditions.

In some embodiments, this lateral routing may reduce or eliminate reactive forces applied to the mask from the supporting surface, such that pressure maintaining the mask against the patient's face may originate primarily from manual application by the healthcare provider. One or more inlets may be selectively capped during use based on provider preference, spatial constraints, or connection requirements.

The respiratory mask 800 may further include an inflatable ring with independently inflatable air chambers 808 and a compliant patient-contacting seal (i.e., seal 606) formed from a thermoplastic elastomer polymer (TEP) material or other deformable materials. These features, alone or in combination, may permit the mask to conform to the patient's facial anatomy under relatively low applied force. In some use scenarios, this configuration may reduce the need for excessive manual pressure to maintain an effective seal and may reduce the likelihood of pressure-related effects during prone positioning.

In some embodiments, the patient-contacting seal may be formed from a thermoplastic elastomer polymer (TEP) material. In other embodiments, the seal may be formed from alternative soft, compliant, or deformable materials capable of conforming to facial anatomy under light manual pressure. Such materials may be selected based on functional characteristics, including softness, tack, resilience, recoverability, durability, chemical resistance, and biocompatibility, rather than a specific chemical composition.

By way of non-limiting example, the seal may include or be formed from silicone-based materials, including silicone gels, liquid silicone rubber (LSR), room-temperature-vulcanizing (RTV) silicone, or combinations thereof. In some embodiments, silicone materials may be formulated to provide varying degrees of softness, surface tack, or recovery characteristics.

In other embodiments, the seal may include polyurethane-based materials, including polyurethane gels, elastomeric polyurethanes, or polyurethane foams. Such materials may be selected to provide energy absorption, shape recovery, or controlled deformation under applied pressure.

In further embodiments, the seal may include heavily plasticized polymer materials, including plasticized polyvinyl chloride (PVC), ethylene-vinyl acetate (EVA), or other polymer blends. Plasticizers, oils, resins, or softening agents may be incorporated to adjust flexibility, tackiness, and glass transition temperature. Suitable plasticizers may include, by way of example, mineral oils, paraffinic oils, hydrocarbon resins, citrates, adipates, or combinations thereof.

### Additional Terms and Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise. Features described herein with respect to a particular embodiment may be omitted, substituted, duplicated, reordered, or combined with features of other embodiments to form additional embodiments not expressly illustrated.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent may also include two or more such referents.

It will also be appreciated that embodiments described herein may also include properties and/or features (e.g., components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

The terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition.

Elements described in relation to any embodiment depicted and/or described herein can be combined with elements described in relation to any other embodiment depicted and/or described herein.

The present invention may be embodied in other forms, without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A respiratory mask comprising:
a mask body defining an exterior surface, a perimeter, and an interior cavity configured to cover a patient's nose and mouth; and
an inflatable ring connected to the perimeter of the mask body, the inflatable ring comprising a plurality of air chambers sealed from one another.

2. The respiratory mask of claim 1, wherein the inflatable ring is attached to the perimeter by an adhesive material.

3. The respiratory mask of claim 1, wherein the mask body has a height of under approximately three inches.

4. The respiratory mask of claim 1, further comprising a plurality of independent inflation ports, each inflation port corresponding to one of the air chambers.

5. The respiratory mask of claim 1, wherein each air chamber is separated from an adjacent air chamber by a flexible partition.

6. The respiratory mask of claim 5, wherein the flexible partition is oriented such that adjacent air chambers overlap along a non-orthogonal boundary.

7. The respiratory mask of claim 1, wherein each air chamber is independently inflatable to conform to individual facial proportions.

8. A respiratory mask comprising:
a mask body defining an exterior surface, a perimeter, and an interior cavity configured to cover a patient's nose and mouth;
an inflatable ring connected to the perimeter of the mask body, the inflatable ring comprising a plurality of air chambers sealed from one another; and
at least two gas inlets positioned on opposing lateral portions of the exterior surface.

9. The respiratory mask of claim 8, wherein the mask body has a height of under approximately three inches.

10. The respiratory mask of claim 8, wherein at least one of the gas inlets comprises a threaded connection configured to engage a complementary threaded component.

11. The respiratory mask of claim 8, wherein at least one of the gas inlets is configured to form a friction-fit connection.

12. The respiratory mask of claim 8, further comprising at least one removable cap configured to seal one of the gas inlets.

13. The respiratory mask of claim 8, wherein the inflatable ring comprises a plurality of independent inflation ports disposed on an exterior surface of the inflatable ring.

14. The respiratory mask of claim 8, wherein the mask body comprises a polymer material.

15. A respiratory mask comprising:
a mask body defining an exterior surface, a perimeter, and an interior cavity configured to cover a patient's nose and mouth;
an inflatable ring connected to the perimeter of the mask body, the inflatable ring comprising a plurality of air chambers sealed from one another;
and a patient-contacting seal disposed over at least a portion of the inflatable ring.

16. The respiratory mask of claim 15, wherein the patient-contacting seal comprises a thermoplastic elastomer polymer material or another deformable polymer material.

17. The respiratory mask of claim 15, wherein the patient-contacting seal has a Shore 00 hardness of up to approximately 00-10.

18. The respiratory mask of claim 15, further comprising at least two gas inlets positioned on opposing lateral portions of the exterior surface.

19. The respiratory mask of claim 15, wherein the inflatable ring is attached to the perimeter of the mask body by an adhesive material.

20. The respiratory mask of claim 15, wherein the mask body comprises a polymer material.
